(19) United States
(12) Patent Application Publication
(10) Pub. No.: US 2011/0217359 A1
(43) Pub. Date: Sep. 8, 2011

(54) PHARMACEUTICAL COMPOSITION FOR THE PROPHYLACTIC OR THERAPEUTIC TREATMENT OF DECUBITI AND FIRST DEGREE BURNS AND TREATMENT METHOD

(76) Inventor: Rabie Stephan, Amherst, NY (US)

(21) Appl. No.: 13/042,092

(22) Filed: Mar. 7, 2011

Related U.S. Application Data
(60) Provisional application No. 61/311,560, filed on Mar. 8, 2010.

(51) Int. Cl. A61K 9/70; A61K 31/4164; A61K 31/4196; A61K 31/7048; A61P 17/02; A61P 31/10; A61P 23/02

(52) U.S. Cl. 424/445; 514/399; 514/383; 514/31

(57) ABSTRACT

This invention relates to a pharmaceutical composition and method for preventing and treating decubitus by topical administration of a therapeutic dosage of a composition containing an antioxidant agent, an anesthetic and vasodilating agent, and an antifungal agent emulsified in a pharmaceutically acceptable carrier.

### PRIORITY CLAIM AND RELATED APPLICATIONS

This patent application claims priority to provisional patent application U.S. Ser. No. 61/311,560 filed Mar. 8, 2010. This application is incorporated by reference in its entirety.

### TECHNICAL FIELD

This invention relates to a method and composition for preventing or treating decubitus and first degree burns by topical administration.

### BACKGROUND

Decubitus ulcer, more commonly known as bedsore, pressure sore or trophic ulcer, is the ischemic necrosis and ulceration of skin tissue overlying a bony prominence of the body that has been subjected to prolonged pressure against an object. The peripheral blood vessel of the tissue is clogged by oppression from a contacting area of the body resulting in necrosis of the tissue. In many cases, a long period of treatment is required. In its most serious presentations, decubitus ulcer may affect epidermal tissue, dermal tissue, muscle tissue and bone. These more serious decubitus ulcers require surgical removal and skin grafting for closure.

Quality of patient care, incidence of infection and remediation of chronic conditions have long been a concern for the health care industry. Recently, however, the so-called “national health care crisis” and political-economic controversy surrounding Medicare and Medicaid reimbursement rates to hospitals and care providers has highlighted the concern for this issue. As health care providers, in particular hospitals, are not reimbursed for treatments associated with this chronic condition under insurance or government program guidelines, it becomes imperative for a cost effect preventative solution to be provided to prevent severe economic distress to hospitals, nursing homes, hospices and physicians who are forced to provide patient care for which they will never be reimbursed.

Decubitus is most frequently found in patients who have been bedridden for long periods of time. Thus, this is a persistent problem for the aged or infirmed that have limited or no mobility. This is also a chronic problem for those with severely diminished or totally absent sensation, such as those suffering from debilitation, emaciation or paralysis induced by physical injury or neurologic disorder. Tissues that are especially susceptible to development of decubitus ulcers include those over the sacrovertebral area, pelvic area, lower pelvic and thigh area, ankles, heels and elbows. However, it is not so limited and other sites may also be involved depending upon the positions of the particular patient.

The best treatment for bedsores is prevention by frequent changing of the bedridden patient's position and providing even distribution of the patient's weight through the use of an anti-bed-sore mattress or cushion. This is not always effective, or possible, with every patient. For example, patient discomfort often prevents the repositioning of patients, as well as the physical and locational challenges posed by the use of certain medical equipment. Prior to the present invention, while many dressings and topical agents have been available for use in the treatment of bedsores, they are as a rule all expensive and in many cases totally useless from a clinical standpoint. The present invention is concerned with meeting the need for an inexpensive, highly effective composition for the prevention and treatment of bedsores and other skin afflictions which comprises a mixture of low cost and readily available therapeutic agents.

### SUMMARY

This invention relates to a pharmaceutical composition and method for preventing and treating decubitus by topical administration of a therapeutic dosage of a composition comprising an antioxidant agent present in an amount ranging between 10-40% by weight, a local anesthetic and vasodilating agent present in an amount ranging between 10-50% by weight, and an antifungal agent present in an amount ranging between 10-50% by weight wherein said antioxidant agent, local anesthetic and vasodilating agent, and antifungal agent are emulsified in a pharmaceutically acceptable carrier. In accordance with this invention, there is also provided a method for preventing decubitus in a subject, comprising administering to a subject a topical composition according to this invention.

This invention further relates to a pharmaceutical composition and method for treating first degree burns by topical administration of a therapeutic dosage of a composition comprising an antioxidant agent present in an amount ranging between 10-40% by weight, a local anesthetic and vasodilating agent present in an amount ranging between 10-50% by weight, and an antifungal agent present in an amount ranging between 10-50% by weight wherein said antioxidant agent, local anesthetic and vasodilating agent, and antifungal agent are emulsified in a pharmaceutically acceptable carrier. In accordance with this invention, there is also provided a method for treating first degree burns in a subject, comprising administering to a subject a topical composition according to this invention.

It is an object of the present invention to provide a composition and treatment method for preventing decubitus.

It is an object of the present invention to provide a composition and treatment method for treating first degree burns.

It is yet another object of this invention to provide a novel pharmacological composition that is economical for mass production from the viewpoint of the manufacturer and consumer, thereby making it economically available to the buying public.

Thus, having broadly outlined the more important features of the present invention in order that the detailed description thereof may be better understood, and that the present contribution to the art may be better appreciated, there are, of course, additional features of the present invention that will be described herein and will form a part of the subject matter of the invention. In this respect, before explaining at least one embodiment of the invention in detail, it is to be understood that the invention is not limited in its application to the details of the composition set forth in the following description. The present invention is capable of other embodiments and of being practiced and carried out in various ways. Also it is to be understood that the phraseology and terminology employed herein are for the purpose of description and should not be regarded as limiting.

### PARTICULAR ADVANTAGES OF THE INVENTION

An antioxidant agent promotes cell growth and regeneration of healthy skin tissue. It also has beneficial properties to aid in the treatment or prevention of fungal and other infections.

A local anesthetic and vasodilating agent provides increased blood flow to the tissue, increasing the overall health of the tissue cells and the absorption of the other therapeutic components. The anesthetic properties increase patient comfort and facilitate other patient care such as tissue cleansing and patient repositioning to prevent pressure sore formation.

An antifungal agent provides prophylactic treatment for the prevention of yeast and fungal infections. The affected areas are often prone to moisture and heat, creating an environment for fungus to thrive. Additionally, the presence of fungus or yeast deprives the tissue of nutrients, slowing the cell regeneration or repair processes.

The emulsion paste creates a protective barrier and controlled ambient environment covering the affected areas. The paste layer may potentially maintain more desirable conditions (e.g., temperature, humidity, pore size, enhanced localized blood flow) for transdermal drug transportation at the affected site. Oxygen freely passes through the barrier but microbes such as viruses, bacteria and fungi are unable to enter and infect the compromised tissues. The barrier keeps the affected tissues moist, enhancing normal cellular processes and general health of the tissues.

The synergistic effect of the individual components increases the health of the affected tissues and the absorption of the therapeutic components. Decubitus is prevented at the earliest stages before sores and ulcers form.

### DEFINITIONS OF TERMS USED IN THIS SPECIFICATION

As used in this application, active ingredient is any component comprising either a chemical moiety, cells or cell extract that is necessary for the therapeutic regime of a subject.

As used in this application, inert ingredient means any non-therapeutic material added to the mixture which does not contribute to the prevention or treatment of decubitus and may include, but is not limited to, solid bulk diluents, binders, pharmaceutically acceptable carriers, preservatives and the like.

As used in this application, pharmaceutically acceptable carrier means any bulk medium that functions as a delivery system for the topical administration of the therapeutic or active ingredient components. They may include water, lipids and/or other alcohols. These also may contain adjunct materials such as salts, surfactants, buffers, thickeners, emulsifiers, thickeners, preservatives, coloring agents, stabilizing agents, perfuming agents, or co-solubilizers.

As used in this application, circulatory system means the structure that moves blood and blood components throughout the body of animals, including the heart, blood vessels and lymph vessels.

As used in this application, liquid suspension is any uniform mixture consisting of a liquid containing undissolved solids.

As used in this application, emulsion is any oil and water emulsions where one is dispersed and one is continuous, such as in the form of creams, ointments, liniments (balms), pastes, films or liquids.

### DETAILED DESCRIPTION

This invention relates to a pharmaceutical composition and method for preventing and treating decubitus and first degree burns by topical administration of a therapeutic dosage of a composition comprising an antioxidant agent, an anesthetic and vasodilating agent, and an antifungal agent in a pharmaceutically acceptable carrier which composition is suitable for topical administration wherein the amount of antioxidant agent, local anesthetic and vasodilating agent, and antifungal agent is effective to treat decubiti. For ease of topical administration, it is usually preferred to administer as a pharmaceutical composition which is manufactured by any method which has been well known in the technical field of pharmaceutical science by mixing the active ingredient with one or more pharmacologically acceptable carrier(s). The pharmaceutically acceptable carrier for topical application may be in the form of a spray, mist, aerosol, lotion, cream, aqueous or non-aqueous solution or liquid, gel, ointment, paste, unguent, emulsion or suspension.

The synergistic effect of the individual components increases the health of the affected tissues and the absorption of the therapeutic components. Decubitus is prevented at the earliest stages before sores and ulcers form. Each constituent promotes and enhances the effectiveness of the other. The optimum effect of the combination is only achieved by simultaneous application, or application in a prescribed order. Random use of the individual components does not provide the advantages of the combination taught herein. The antifungal kills competing organisms and is preferably applied first or simultaneously with the other components. An antifungal agent provides prophylactic treatment for the prevention of yeast and fungal infections. The affected areas are often prone to moisture and heat, creating an environment for fungus to thrive. Additionally, the presence of fungus or yeast deprives the tissue of nutrients, slowing the cell regeneration or repair processes.

The local anesthetic and vasodilating agent is preferably applied second or simultaneously with the other components. A local anesthetic and vasodilating agent provides increased blood flow to the tissue, increasing the overall health of the tissue cells and the absorption of the other therapeutic components. Lastly, or simultaneously, is applied an antioxidant agent that promotes cell growth and regeneration of healthy skin tissue. It also has beneficial properties to aid in the treatment or prevention of fungal and other infections.

There is an effective, though not excessive, amount of antioxidant agent. Preferably, the antioxidant agent is present in an amount ranging between 10% and 40% by weight. There is an effective, though not excessive, amount of local anesthetic and vasodilating agent. Preferably, the anesthetic agent is present in an amount ranging between 10% and 50% by weight. There is an effective, though not excessive, amount of antifungal agent. Preferably, the antifungal agent is present in an amount ranging between 10% and 50% by weight.

There is present each component in an amount that is enough to be therapeutically effective and below thresholds for toxicity.

In a preferred embodiment, the composition is an emulsion of the three active agents. The emulsion in a paste form creates a protective barrier and controlled ambient environment covering the affected areas. The paste layer may potentially maintain more desirable conditions (e.g., temperature, humidity, pore size, enhanced localized blood flow) for transdermal drug transportation at the affected site.

In a preferred simple and effective embodiment, the composition comprises a paste formed of A & D ointment, a local anesthetic and an antifungal. A preferred composition and method for carrying out the present invention is illustrated by the following example:

A paste is formed with substantially equal parts A and D ointment, miconozol and lidocaine. The paste is applied to a portion of the skin prone to decubitus by rubbing a small amount and letting it dry on the skin surface to provide a protective prophylactic barrier against the formation of decubitus. The emulsion paste is applied topically to the affected area at the first signs of skin compromise such as excessive warmth to the touch or redness.

To treat early stage decubitus, the thickened mixture is applied directly to the bedsore or otherwise affected skin area and the area maintained in a moistened condition. If desired, a cover may be applied such as a bandage, gauze, polymeric cover or the like. While the preferred mode of application is to daube a paste mixture directly onto the affected skin area, the compositions of the present invention may also be sprayed or combined with suitable pharmaceutical carriers or bases for application. The compositions of the present invention may also be applied to a bandage or cover that is adhered to the skin such that the composition is applied to the affected skin site for surface and/or transdermal delivery. The composition may also be in the form of a solid ulcer dressing having the compound dispersed on or in the dressing. The way to prepare this embodiment is well known in the art.

Local Anesthetic and Vasodilating Agent

Dilation of the local blood vessels will result in an increase in blood flow to the treated area of skin. The transiently improved blood flow to the treated area should improve the metabolic condition of the tissue, which in turn should result in a healthier condition for nerve and associated tissue function. Improving the metabolic state around the nerve cells in the skin through an improved blood flow, which would increase the oxygenation and nutrient delivery to the tissue as well as improve the removal of cellular and tissue waste products, should improve the health and functioning of the surrounding tissue cells.

The anesthetic properties increase patient comfort and facilitate other patient care such as tissue cleansing and patient repositioning to prevent pressure sore formation.

The concentration of anesthetic and vasodilating agent in the compositions described herein can be therapeutically effective, meaning the concentration is adequate to provide a therapeutic benefit without inflicting harm to the patient. The compositions described herein may have an anesthetic and vasodilating agent concentration of between about 10% and about 50% weight/volume (w/v) of the composition. Preferably, the anesthetic and vasodilating agent concentration is between about 33% and about 40% weight/volume (w/v) of the composition.

The compositions described herein include a therapeutically effective amount of at least one local anesthetic and vasodilating agent. In one embodiment, the at least one anesthetic agent is lidocaine, such as in the form of lidocaine HCl. Lidocaine as used herein refers to lidocaine HCl and other forms of lidocaine which can be useful as topical anesthetics. In another embodiment, the at least one anesthetic agent is tetracaine. In yet another embodiment, a combination of lidocaine and tetracaine is used.

As will be appreciated, other local anesthetic and vasodilating agents may be adapted to perform the intended function. Other amino amides that are used with the present invention include mepivacaine, prilocaine, bupivacaine, etidocaine, and ropivacaine and levobupivacaine. In some embodiments, amino esters are used, such as, for example, cocaine, procaine, tetracaine, chloroprocaine, and benzocaine. A combination of tetracaine and lidocaine is therapeutically and cost effective and is sold under the trade name PLIAGLIS.™

The at least one local anesthetic can also be selected from the group of ambucaine, amolanone, amylocaine, benoxinate, betoxycaine, biphenamine, butacaine, butamben, butanilicaine, butethamine, butoxycaine, carticaine, cocaethylene, cyclomethycaine, dibucaine, dimethysoquin, dimethocaine, diperodon, dycyclonine, ecgonidine, ecgonine, ethyl chloride, beta-eucaine, euprocin, fenalcomine, formocaine, hexylcaine, hydroxytetracaine, isobutyl p-aminobenzoate, leucinocaine mesylate, levoxadrol, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, propanocaine, proparacaine, propipocaine, propoxycaine, psuedococaine, pyrrocaine, salicyl alcohol, tolycaine, trimecaine, zolamine, and salts thereof.

Anesthetic Enhancers

Cations, such as K+, Mg++, and H+, can be added to enhance the effects of the lidocaine.

Other Vasodilators

The local anesthetic agent generally provides a vasodilating effect. However, in some alternate embodiments, these functions may be provided by separate compounds or a combination of compounds. Other vasodilators that are candidates as a single mode therapeutic or in combination with other vasodilators, which may be suitably used with the present invention as a direct vasodilator or those substances that serve as precursors or second messenger chemicals to induce or enhance the production of vasodilatory substance from the tissue and cell. Examples of chemical vasodilators include, by example only but are not limited to: amrinone, L-arginine, bamethan sulphate, bencyclane fumarate, benfurodil hemisuccinate, benzyl nicotinate, buflomedil hydrochloride, buphenine hydrochloride-, butalamine hydrochloride, cetiedil citrate, ciclonicate, cinepazide maleate, cyclandelate, di-isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha-adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion-blocking agents include pentolinium and trimetaphan. Calcium channel blockers include amlodipine, diltiazem, felodipine, isradipine, nicardipine, nifedipine, nimodipine, and vera-pamil. Prostaglandins including: prostacyclin, thrombuxane A2, leukotrienes, PGA, PGA1, PGA2, PGE1, PGE2, PGD, PGG, and PGH. Angiotensin II analogs include saralasin. Other vasodilators include nitroglycerin, labetalol, thrazide, isosorbide dinitrate, pentaerythritol tetranitrate, digitalis, hydralazine, diazoxide, and sodium nitroprusside. Typically the vasodilator, used either as a single agent or in concert with other vasodilators is present in the topical vehicle at concentration between 10 to 50%, depending on the specific vasodilator used and the pharmacologic properties of the chemical.

Antioxidant Agent

The antioxidant agent is preferably an antioxidant agent selected from a group consisting of vitamin E, vitamin C, vitamin A, pyconogenol, A&D ointment, and combinations thereof. Preferably, at least one of vitamins A, C, D and E is provided for its known antioxidant and skin healing properties. More preferably, a combination of two or more of vitamins A, C, D and E is provided in the composition. Enhancing the general health of the tissue allows the other therapeutic components to be more effective. Healthy tissues boosts the resistive properties of the cells against cellular damage.

Fish oils, and especially cod liver oil, provide skin healing and softening properties. Cod liver oil provides a source of antioxidants, in particular, vitamins A and D. One milliliter of cod liver oil contains approximately 850 IU of vitamin A and 85 IU of vitamin D within a rich source of omega-3 fatty acids.

Other antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl fuirfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

The concentration of an antioxidant agent in the compositions described herein can be therapeutically effective meaning the concentration is adequate to provide a therapeutic benefit without inflicting harm to the patient. The compositions described herein may have an antioxidant agent concentration of between about 10% and about 40% weight/volume (w/v) of the composition. Preferably, the anesthetic and vasodilating agent concentration is between about 20% and about 33% weight/volume (w/v) of the composition.

Fatty Acid Source

In some embodiments, the composition further comprises a fatty acid source. Fatty acids, among other things, function as penetration enhancers to assist in the transport of the vasodilators from the skin surface, through the stratum corneum and into the dermal layer of the skin. Suitable fatty acids include by example but are not limited to: linoleic acids, linolenic acids, oleic acids, stearic acids, and myristic acids. Numerous fish oils, free fatty acids, triglicerides, or omega-3 fatty acid oils can be used, ideally if they contain natural trace minerals.

Antifungal Agent

The antifungal agent is used topically for the prevention and treatment of certain dermatophytoses, such as Tinea cruris, Tinea corporis, Tinea manuum, and Tinea pedis, which are caused by Trychophyton rubrum, Trychophyton tonsurans, Microsporum canis, Microsporum audouini, or Epidermophyton floccosum. Other fungal and yeast infections may also be treated.

Affected areas are often prone to excessive moisture and heat, allowing fungal and yeast infections to thrive. Growth of these infections decreases available nutrients for healthy cell functions. Thus, prevention and treatment of these microbes provides healthier tissues that can absorb the other therapeutics, self-repair or defend against degradation and infection. For example, tissues with fungal infections are often excessively dry, making them difficult to treat effectively.

Preferably, the antifungal agent is an antifungal agent selected from a group consisting of micocnozol, fluconozol, nystatin and combinations thereof. It is to be understood that the antifungal agents miconozol, nystatin or fluconozol are only preferred examples of the antifungal agents that can be utilized with this invention.

Other antifungal agents suitable for the invention include imidazoles, traizoles, allylamines, and mixtures thereof. Suitable imidazoles include miconazole, ketoconazole, clotrimazole, econazole, mebendazole, bifonazole, butoconazole, fenticonazole, isoconazole, oxiconazole, sertaconazole, sulconazole, thiabendazole, and tiaconazole. Suitable triazoles include fluconazole, itraconazole, ravuconazole, and posaconazole. Suitable allylamines include terbinafine, amorolfine, naftifine, and butenafine, including the salts thereof. All of the antigungal agents can be in a pharmaceutically active salt form. Examples of suitable salt forms are the hydrochloride, lactate and ascorbate forms.

Particularly preferred antifungal agents are allylamines and salts thereof. Other antifungal agents, whether or not presently approved by the U.S. Food & Drug Administration, may be selected and used, once approved and shown effective.

As will be described in greater detail below, antioxidant agents and fatty acids are other desirable components of the composition. Fish oils are a good source of both of these, and are the source in the example provided using A and D ointment. Antifungals that combine well with fish oils include polyenes such as nystatin; imidazoles such as clortimazole, econazole, ketoconazole, miconazole, solconazole, and oxiconizole; and, allylamines-benzylamines, including naftifine, terbinafine, and butenafine, for example. Other antifungals such as tolnaftate, triacetin, and grisiofulvin may also be suitably used.

Vitamin A and vitamin E in high concentrations may possess some antifungal properties by aiding the body heal itself. Vitamins A and E, with fatty acids, add synergy in treating fungal infections.

Herbal supplements, such as garlic or garlic oil, for example, are incorporated into certain embodiments, for their skin healing and fungistatic and/or fungicidal properties by organic acids and volatile oils, contained within garlic oil, including organosulfur compounds, phytic acid, saponins, amino acids such as arginine.

Similarly, xylitol is incorporated in some embodiments for its antibacterial, antifungal, dermal hydration and moisture affinity properties.

The concentration of antifungal agent in the compositions described herein can be therapeutically effective meaning the concentration is adequate to provide a therapeutic benefit without inflicting harm to the patient. The compositions described herein may have an antifungal agent concentration of between about 10% and about 50% weight/volume (w/v) of the composition. Preferably, the antifungal agent concentration is between about 33% and about 40% weight/volume (w/v) of the composition.

Method of Administration

The topical compositions according to the invention are preferably dermatological compositions applied topically to the skin. The route of administration of the composition of the present invention is topical and applied at ambient temperatures. The composition of the present invention should not be injected instead of being applied topically. This effective amount of the composition may be administered as a topical composition at a given frequency, such as about once a week, about twice a week, about three times a week, once a day, about twice a day, about three times a day, and the like. Preferably, the effective amount is administered two to four times per day, at least 4 hours apart, over a prolonged period of time, e.g. days, weeks or months.

The effective amount of the novel composition of this invention and frequency of administration may depend on a variety of factors, such as the components utilized, the general health of the subject being treated, and the physiological and dermatological characteristics of the subject being treated.

The paste emulsion, when applied and left to dry on the skin surface, creates a controlled suitable microenvironment at the administration site to facilitate the delivery of drugs across the skin. Oxygen freely passes through the barrier but microbes such as viruses, bacteria and fungi are unable to enter and infect the compromised tissues. The barrier also keeps the affected tissues moist, enhancing normal cellular processes and general health of the tissues.

Too cold an environment can result in little blood supply to the dermal barrier; pores and other natural openings in the dermal barrier constrict, thereby preventing efficient transport. Too hot an environment can enhance secretion and perspiration and vapor flow through the dermal barrier, creating negative conditions for transdermal drug delivery activity. Too dry an environment can cause an element or elements of the delivery complex to evaporate quickly, losing its ability to transport. The enhanced evaporation also creates negative transport pressure. Too humid an environment can cause dilution of the active ingredient, diminishing the capacity of the active ingredient and also creating negative transport activity. The paste layer may potentially maintain more desirable conditions (e.g., temperature, humidity, pore size, enhanced localized blood flow) for transdermal drug transportation at the affected site.

Pharmaceutically Acceptable Carriers

The active ingredients may be mixed as a paste to form a simple topical composition according to this invention. This may be applied directly to the skin. A paste emulsion is prepared by combining the pharmaceutically active components with a carrier such as the base used with a typical A and D ointment: a combination of one or more skin protecting agents from the group of lanolin, paraffin (microcrystalline wax), petrolatum, mineral oil, and aloe. (Where and actual A and D ointment is used, the antioxidant agent is provided therein.)

In other embodiments, more complex compositions may be formed with other components. Some of these components will be described below in greater detail.

The topical composition can also advantageously be in liquid or gel form.

Lidocaine base is freely lipid soluble. It is insoluble in water and thus not suitable for use in an aqueous suspension, requiring ethanol or the like to obtain a liquid solution. Examples of organic solvents considered useful in these formulations are ethyl alcohol, benzyl alcohol, propylene glycol, diethyl ether, dimethoxyethane, etc.

The compositions according to the invention can also be made available in any form known in the field of cosmetology including creams, emulsions, milks, sprays, solutions (both aqueous and hydro-alcoholic), anhydrous bases, gels, ointments, moisturizing cream, skin benefit creams and lotions, or by other method or any combination of the forgoing as would be known to one of ordinary skill in the art. These might be especially suitable for home care use where institutional grade pharmaceuticals are not readily accessible or may be less likely to be used.

The composition of cosmetic applications is well known in the art. These compositions can also contain one or more formulation agents or additives of known and conventional use in cosmetic and dermatological compositions, including but not limited to softeners, colorants, film-forming agents, surface-active agents, perfumes, preservatives, emulsifiers, oils, glycols, sebum-absorption agents, vitamins and the like. Those skilled in the cosmetics art know which formulation agents to add to the compositions of the invention and in what quantities in relation to the desired properties. It is important, however, that the concentrations and combinations of the compounds and extracts be selected in such a way that the combinations are chemically compatible and do not form complexes which precipitate from the finished product. It is important that the additional components of the cosmetic composition do not impair the functionality of the active ingredients.

Other Compounds

Compositions of the present invention can include other beneficial agents and compounds that are non-toxic and pharmaceutically acceptable compounds. The composition may further comprise a component selected from the group consisting of buffering agents, emulsifying agents, thickeners, solvents, preservatives, coloring agents, surfactants, stabilizing agents, perfuming agents, or co-solubilizers.

In some embodiments, the carrier will also include chemicals that function as penetration enhancers to assist in the transport of the vasodilators from the skin surface, through the stratum corneum and into the dermal layer of the skin. Suitable enhancers include by example only but are not limited to: individual fatty acids, fatty acid esters, polyols, amides, various anionic, cationic and nonionic surfactants such as but not limited to sodium laurate and sodium lauryl sulfate, phospholipids, cholesterol and cholesterol derivatives, m-pyrrole, dimethyl acetamide, limonene, sphingolipids, ceramides, terpenes, alkanones, menthol, various organic acids, such as but not limited to salicylic acid, citric and succininc acid, prostaglandins, decyl methyl sulfoxide, urea, sulfoxide alcohols, plant extract oils.

Phospholipids include by example but are not limited to: phosphatidylcholine, phosphatidylethanolamine, and phosphatidylserine.

Plant extract oils include peanut oil, hemp, barag, olive oil, sunflower oil, soybean oil, monoi oil and macadamia oil, with olive oil being preferred.

Suitable alcohols for the plant extract oil/alcohol mix include ethyl alcohol, isopropyl alcohol, methyl alcohol and witch hazel.

Other additives that can be used with the compositions of the present invention include honey, various herb extracts, Aloe Barbadensis (Organic Aloe Vera) Leaf Juice, silk or soy amino acids, other amino acids, surfactants, proteins, fragrance oils, essential oils, Nasturtium Officinate (Watercress) Extract, and Chamomilla Recutilla (Matricaria) Extract.

A drying agent that can be sued is zinc oxide or aluminum oxide.

Amino acids may be incorporated to aid in absorption into the skin, as well as their healing properties.

Alcohol, preferably ethyl alcohol, may be used to increase the solubility of other agents of the composition. Alcohol also aids in combating the growth of the infecting fungi.

Acidifiers that can be used with the compositions of the present invention include ascorbic acid, citric acid, dihydroxytartaric acid, glutaric acid, iodacetic acid, itaconic acid, malic acid, mandelic acid, oxalic acid, salicylic acid, succinic acid and a- and meso-tartaric acid. The most preferred acid is citric acid, especially in its anhydrous form. Other useful acid compounds are alkali metal and ammonium salts thereof, e.g. monosodium citrate, monosodium tartrate and potassium tetroxalate.

Moisturizing agents that can be used with the compositions of the present invention include xylitol, amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays)oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (asminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffm, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-1 50 stearate, pentadecalactone, peppermint (mentha piperita) oil, petrolatum, phospholipids, polyamino sugar condensate, polyglyceryl-3 diisostearate, polyquatemium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quatemium-15, quatemium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

Thickeners and emulsifiers that can be used with the compositions of the present invention include ammonium xylenesulfonate, Glycerol stearate, Sodium chloride (table salt), amonium chloride and modified cellulose based thickeners.

Foaming agents that can be used with the compositions of the present invention include Cocamide DEA (or MEA or TEA).

A wax that can be used with the compositions of the present invention include glycol distearate.

Preservatives that can be used with the compositions of the present invention include DMDM hydantoin, imidazolidinyl urea, biocides, isothiazolinone, BHA, methylisothiazolinone, methylchloroisothiazolinone, Sodium benzoate, 2-bromo-2-nitropropane-1,3-diol.

Buffers that can be used with the compositions of the present invention include sodium citrate.

Colors and dyes that can be used with the compositions of the present invention include Yellow 5 (Cl#19140), Urea, Red 33 (Cl#17200), Blue 1 (Cl#42090), Green 5, Ext. Violet 2, Green 8, Red 40, Yellow 6, D&C Blue no. 2, D&C Red no. 27, D&C Red no. 21, D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate.

Numerous modifications and variations of the present invention are possible in light of the above teachings and therefore within the scope of the appended claims, and the invention may be practiced otherwise than as particularly described.

Treatment of First Degree Burns

The composition and treatment method disclosed herein are also advantageously used in the treatment of first degree burns. This course of treatment is not advantageously used with second, third or other degrees of skin burns.

The method for the therapeutic treatment of first degree burns comprises the step of topically applying to the affected skin tissue an effective amount of a composition comprising an antioxidant agent, a local anesthetic and vasodilating agent, and an antifungal agent in a pharmaceutically acceptable carrier. Preferably, although not required in all embodiments, the antifungal agent, antioxidant agent, local anesthetic and vasodilating agent thereof are present in equal amounts in the composition. Preferably, the antioxidant agent thereof is an antioxidant agent selected from a group consisting of vitamin E, vitamin C, pyconogenol, A&D ointment, and combinations thereof. Preferably, the antifungal agent thereof is an antifungal agent selected from a group consisting of micocnozol, fluconozol, nystatin and combinations thereof. Preferably, the local anesthetic and vasodilating agent thereof is a local anesthetic and vasodilating agent selected from a group consisting of lidocaine, tetracaine and combinations thereof.

1. A pharmaceutical composition for the prophylactic or therapeutic treatment of decubiti ulcers and first degree burns consisting of an antioxidant agent, a local anesthetic and vasodilating agent, an antifungal agent and a pharmaceutically acceptable carrier, which composition is suitable for topical administration wherein the amount of antioxidant agent, local anesthetic and vasodilating agent, and antifungal agent is effective to treat decubiti.

2. The pharmaceutical composition of claim 1 wherein said pharmaceutically acceptable carrier for topical application is in the form of a spray, mist, aerosol, lotion, cream, aqueous or non-aqueous solution or liquid, gel, ointment, paste, unguent, emulsion or suspension.

3. The pharmaceutical composition of claim 1 wherein the composition is in the form of a solid ulcer dressing having the composition dispersed on or in the dressing.

4. The pharmaceutical composition of claim 1 further comprising at least one component selected from the group consisting of buffers, emulsifiers, thickeners, preservatives, coloring agents, surfactants, stabilizing agents, perfuming agents, or co-solubilizers.

5. The pharmaceutical composition of claim 1 further comprising at least one fatty acid.

6. The pharmaceutical composition of claim 1 wherein the antioxidant agent thereof is from 10% to 34% w/v of the composition.

7. The pharmaceutical composition of claim 1 wherein the antifungal agent thereof is from 10% to 40% w/v of the composition.

8. The pharmaceutical composition of claim 1 wherein the local anesthetic and vasodilating agent thereof is from 10% to 50% w/v of the composition.

9. The pharmaceutical composition of claim 1 wherein the antifungal agent, antioxidant agent, local anesthetic and vasodilating agent thereof are present in equal amounts in the composition.

10. The pharmaceutical composition of claim 1 wherein the antioxidant agent thereof is an antioxidant agent selected from a group consisting of vitamin E, vitamin C, vitamin A, pyconogenol, A&D ointment, and combinations thereof.

11. The pharmaceutical composition of claim 1 wherein the antifungal agent thereof is an antifungal agent selected from a group consisting of micocnozol, fluconozol, nystatin and combinations thereof.

12. The pharmaceutical composition of claim 1 wherein the local anesthetic and vasodilating agent thereof is a local anesthetic and vasodilating agent selected from a group consisting of lidocaine, tetracaine and combinations thereof.

13. The pharmaceutical composition of claim 9 wherein the antifungal agent thereof is fluconozol, the antioxidant agent is A&D ointment and the local anesthetic and vasodilating agent is lidocaine.

14. The pharmaceutical composition of claim 9 wherein the antifungal agent thereof is nystatin, the antioxidant agent is A&D ointment and the local anesthetic and vasodilating agent is lidocaine.

15. The pharmaceutical composition of claim 9 wherein the antifungal agent thereof is micocnozol, the antioxidant agent is A&D ointment and the local anesthetic and vasodilating agent is lidocaine, tetracaine or a combination thereof.

16. The pharmaceutical composition of claim 9 wherein the antifungal agent thereof is micocnozol, the antioxidant agent is pycnogenol and the local anesthetic and vasodilating agent is lidocaine.

17. The pharmaceutical composition of claim 1 wherein the antifungal agent thereof is micocnozol, the antioxidant agent is vitamin E and the local anesthetic and vasodilating agent is lidocaine, and wherein the antifungal agent , the antioxidant agent, and the local anesthetic and vasodilating agent thereof are present in a ratio of 2:1:2 (antifungal:antioxidant:local anesthetic and vasodilator) in the composition.

18. A method for the prophylatic or therapeutic treatment of decubiti, the method comprising the step of topically applying to decubiti an effective amount of a composition comprising an antioxidant agent, a local anesthetic and vasodilating agent, and an antifungal agent in a pharmaceutically acceptable carrier.

19. The method for the prophylatic or therapeutic treatment of decubiti of claim 18 wherein the antifungal agent , antioxidant agent, local anesthetic and vasodilating agent thereof are present in equal amounts in the composition.

20. The method for the prophylatic or therapeutic treatment of decubiti of claim 18 wherein the antioxidant agent thereof is an antioxidant agent selected from a group consisting of vitamin E, vitamin C, pyconogenol, A&D ointment, and combinations thereof.

21. The method for the prophylatic or therapeutic treatment of decubiti of claim 18 wherein the antifungal agent thereof is an antifungal agent selected from a group consisting of micocnozol, fluconozol, nystatin and combinations thereof.

22. The method for the prophylatic or therapeutic treatment of decubiti of claim 18 wherein the local anesthetic and vasodilating agent thereof is a local anesthetic and vasodilating agent selected from a group consisting of lidocaine, tetracaine and combinations thereof.

23. A method for the therapeutic treatment of first degree burns, the method comprising the step of topically applying to the affected skin tissue an effective amount of a composition comprising an antioxidant agent, a local anesthetic and vasodilating agent, and an antifungal agent in a pharmaceutically acceptable carrier.

24. The method for the therapeutic treatment of first degree burns of claim 23 wherein the antifungal agent, antioxidant agent, local anesthetic and vasodilating agent thereof are present in equal amounts in the composition.

25. The method for the therapeutic treatment of first degree burns of claim 23 wherein the antioxidant agent thereof is an antioxidant agent selected from a group consisting of vitamin E, vitamin C, pyconogenol, A&D ointment, and combinations thereof.

26. The method for the therapeutic treatment of first degree burns of claim 23 wherein the antifungal agent thereof is an antifungal agent selected from a group consisting of micocnozol, fluconozol, nystatin and combinations thereof.

27. The method for the therapeutic treatment of first degree burns of claim 23 wherein the local anesthetic and vasodilating agent thereof is a local anesthetic and vasodilating agent selected from a group consisting of lidocaine, tetracaine and combinations thereof.
